(19) United States
(12) Patent Application Publication
(10) Pub. No.: US 2014/0072622 A1
(43) Pub. Date: Mar. 13, 2014

(54) VACCINE AGAINST STREPTOCOCCUS PNEUMONIAE

(71) Applicants: Philippe Denoel, Rixensart (BE); Jan Poolman, Haarlem (NL); Vincent Verlant, Rixensart (BE); Hugues Wallemacq, Rixensart (BE)

(72) Inventors: Philippe Denoel, Rixensart (BE); Jan Poolman, Haarlem (NL); Vincent Verlant, Rixensart (BE); Hugues Wallemacq, Rixensart (BE)

(73) Assignee: GLAXOSMITHKLINE BIOLOGICALS S.A., Rixensart (BE)

(21) Appl. No.: 14/118,387

(22) PCT Filed: May 15, 2012
(86) PCT No.: PCT/EP2012/058987

§ 371 Date: Nov. 18, 2013

(30) Foreign Application Priority Data
May 17, 2011 (GB) 1108256.7
Dec. 16, 2011 (GB) 1121647.0

(51) Int. Cl. A61K 39/09

(52) U.S. Cl. CPC A61K 39/092
USPC 424/450; 424/244.1

(57) ABSTRACT

The present invention relates to improved immunogenic compositions and vaccines, methods for making them and their use in medicine. In particular the invention relates to immunogenic compositions of unconjugated Streptococcus pneumoniae proteins selected from: pneumolysin and member(s) of the Polyhistidine Triad family (e.g. PhtD), which comprise adjuvants comprising QS21 and monophosphoryl lipid A (MPL), and are presented in the form of a liposome.

### TECHNICAL FIELD

The present invention relates to improved immunogenic compositions and vaccines, methods for making them and their use in medicine. In particular the invention relates to immunogenic compositions of unconjugated Streptococcus pneumoniae proteins selected from: pneumolysin and member(s) of the Polyhistidine Triad family (e.g. PhtD), which comprise adjuvants comprising QS21 and monophosphoryl lipid A (MPL), and are presented in the form of a liposome.

### TECHNICAL BACKGROUND

Streptococcus pneumonia (S. pneumoniae), also known as the pneumococcus, is a Gram-positive bacterium. S. pneumoniae is a major public health problem all over the world and is responsible for considerable morbidity and mortality, especially among infants, the elderly and immunocompromised persons. S. pneumoniae causes a wide range of important human pathologies including community-acquired pneumonia, acute sinusitis, otitis media, meningitis, bacteremia, septicemia, osteomyelitis, septic arthritis, endocarditis, peritonitis, pericarditis, cellulitis, and brain abscess. S. pneumoniae is estimated to be the causal agent in 3,000 cases of meningitis, 50,000 cases of bacteremia, 500,000 cases of pneumonia, and 7,000,000 cases of otitis media annually in the United States alone (Reichler, M. R. et al., 1992, J. Infect. Dis. 166: 1346; Stool, S. E. and Field, M. J., 1989 Pediatr. Infect. Dis J. 8: S11). Mortality rates due to pneumococcal disease are especially high in children younger than 5 years of age from both developed and developing countries. The elderly, the immunocompromised and patients with other underlying conditions (diabetes, asthma) are also particularly susceptible to disease.

The major clinical syndromes caused by S. pneumoniae are widely recognized and discussed in all standard medical textbooks (Fedson D S, Muscher D M. In: Plotkin S A, Orenstein W A, editors. Vaccines. Orth edition. Philadelphia WB Saunders Co, 2004a: 529-588). For instance, Invasive pneumococcal disease (IPD) is defined as any infection in which S. pneumoniae is isolated from the blood or another normally sterile site (Musher D M. Streptococcus pneumoniae. In Mandell G L, Bennett J E, Dolin R (eds). Principles and Practice of Infectious diseases (5th ed). New York, Churchill Livingstone, 2001, p 2128-2147).

Chronic obstructive pulmonary disease is a chronic inflammatory disease of the lungs and a major cause of morbidity and mortality worldwide. Approximately one in 20 deaths in 2005 in the US had COPD as the underlying cause. (Drugs and Aging 26:985-999 (2009)). It is projected that in 2020 COPD will rise to the fifth leading cause of disability adjusted life years, chronic invalidating diseases, and to the third most important cause of mortality (Lancet 349:1498-1504 (1997)).

The course of COPD is characterized by progressive worsening of airflow limitation and a decline in pulmonary function. COPD may be complicated by frequent and recurrent acute exacerbations (AE), which are associated with enormous health care expenditure and high morbidity. (Proceedings of the American Thoracic Society 4:554-564 (2007)). One study suggests that approximately 50% of acute exacerbations of symptoms in COPD are caused by non-typeable Haemophilus influenzae, Moraxella catarrhalis, Streptococcus pneumoniae, and Pseudomonas aeruginosa. (Drugs and Aging 26:985-999 (2009)). H. influenzae is found in 20-30% of exacerbations of COPD; Streptococcus pneumoniae, in 10-15% of exacerbations of COPD; and Moraxella catarrhalis, in 10-15% of exacerbations of COPD. (New England Journal of Medicine 359:2355-2365 (2008)). Haemophilus influenzae, Streptococcus pneumoniae, and Moraxella catarrhalis have been shown to be the primary pathogens in acute exacerbations of bronchitis in Hong Kong, South Korea, and the Phillipines, while Klebsiella spp., Pseudomonas aeruginosa and Acinetobacter spp. constitute a large proportion of pathogens in other Asian countries/regions including Indonesia, Thailand, Malaysia and Taiwan (Respirology, (2011) 16, 532-539; doi:10.1111/j.1440.1843.2011.01943.x). In Bangladesh, 20% of patients with COPD showed positive sputum culture for Pseudomonas, Klebsiella, Streptococcus pneumoniae and Haemophilus influenzae, while 65% of patients with AECOPD showed positive cultures for Pseudomonas, Klebsiella, Acinetobacter, Enterobacter, Moraxella catarrhalis and combinations thereof. (Mymensingh Medical Journal 19:576-585 (2010)). However, it has been suggested that the two most important measures to prevent COPD exacerbation are active immunizations and chronic maintenance of pharmacotherapy. (Proceedings of the American Thoracic Society 4:554-564 (2007)).

Although the advent of antimicrobial drugs has reduced the overall mortality from pneumococcal disease, the emergence of antibiotic resistant strains of S. pneumoniae is a serious and rapidly increasing problem. It is therefore important for effective vaccines against S. pneumoniae to be developed. Effective pneumococcal vaccines could have a major impact on the morbidity and mortality associated with S. pneumoniae disease.

The present invention relates to immunogenic compositions of unconjugated S. pneumoniae proteins presented in the form of a liposome. Liposome formulations are known in the art, and have been suggested to be useful as adjuvant compositions (WO96/33739, WO07/068,907). WO96/33739 discloses certain vaccines containing an antigen, an immunologically active fraction derived from the bark of Quillaja Saponaria Molina such as QS21, and a sterol, which may be presented in the form of a liposome, and methods for the preparation of liposomes. WO07/068,907 discloses certain immunogenic compositions comprising an antigen or antigenic preparation, in combination with an adjuvant which comprises an immunologically active saponin fraction derived from the bark of Quillaja Saponaria Molina presented in the form of a liposome and a lipopolysaccharide where the saponin fraction and lipopolysaccharide are both present in a human dose as a level below 30 μg.

However, there is still a need for improved vaccine compositions, particularly ones which will be more effective in the prevention or amelioration of pneumococcal diseases in the elderly and in young children. The present invention provides an improved vaccine based on a specific combination of unconjugated S. pneumoniae proteins and adjuvants.

### STATEMENT OF THE INVENTION

The present inventors have discovered vaccine or immunogenic compositions of unconjugated Streptococcus pneumoniae proteins selected from: pneumolysin and member(s) of the Polyhistidine Triad family (e.g. PhtD), in combination with an adjuvant comprising QS21, monophosphoryl lipid A (MPL), phospholipid and sterol, presented in the form of a liposome have advantageous properties. This combination of unconjugated S. pneumoniae proteins and adjuvant has been found to provide enhanced immunogenic responses.

Accordingly, in the first aspect of the present invention there is provided an immunogenic composition comprising at least one unconjugated S. pneumoniae protein selected from: pneumolysin and member(s) of the Polyhistidine Triad family (e.g. PhtD); and an adjuvant comprising QS21, monophosphoryl lipid A (MPL), phospholipid and sterol, presented in the form of a liposome.

In another aspect of the present invention, there is provided a vaccine composition comprising at least one unconjugated S. pneumoniae protein selected from: pneumolysin and member(s) of the Polyhistidine Triad family (e.g. PhtD); and an adjuvant comprising QS21, monophosphoryl lipid A (MPL), phospholipid and sterol, presented in the form of a liposome.

In a further aspect of the invention there is provided a method of treating or preventing a disease caused by Streptococcus pneumoniae infection comprising intramuscularly administering to a subject in need thereof comprising administering to said subject an immunogenic composition comprising at least one unconjugated S. pneumoniae protein selected from: pneumolysin and member(s) of the Polyhistidine Triad family (e.g. PhtD); and an adjuvant comprising QS21, monophosphoryl lipid A (MPL), phospholipid and sterol, presented in the form of a liposome.

In a further aspect of the invention there is provided the use of an immunogenic composition comprising at least one unconjugated S. pneumoniae protein selected from: pneumolysin and member(s) of the Polyhistidine Triad family (e.g. PhtD); and an adjuvant comprising QS21, monophosphoryl lipid A (MPL), phospholipid and sterol, presented in the form of a liposome, in the manufacture of a medicament for use in treating or preventing a disease caused by S. pneumoniae infection.

### BRIEF DESCRIPTION OF FIGURES

FIG. 1: Overall dPly specific T cells response in blood: AS03B vs AS01B. T cells expressing any cytokines (IFN-g, IL-2, IL-17, IL-13) at PIII (i.e. after the third immunization).

FIG. 2: Overall PhtD specific T cells response in blood: AS03B vs AS01B. T cells expressing any cytokines (IFN-g, IL-2, IL-17, IL-13).

FIG. 3: dPly specific Th1 response: AS03B vs AS01B. IFNg-expressing T cells (Th1).

FIG. 4: PhtD specific Th1 response: AS03B vs AS01B. IFNg-expressing T cells (Th1).

FIG. 5: dPly specific Th17 response: AS03B vs AS01B PIII

FIG. 6: PhtD specific Th17 response AS03B vs AS01B

FIG. 7: AS01B vs AS03B: antibody response. FIG. 7a: PhtD dosage IgG total. FIG. 7b: dPly Dosage IgG total.

FIG. 8: Evaluation of AS01B and AS01E in the lethal challenge model.

FIG. 9: Evaluation of AS01B and AS01E in the lung colonisation model.

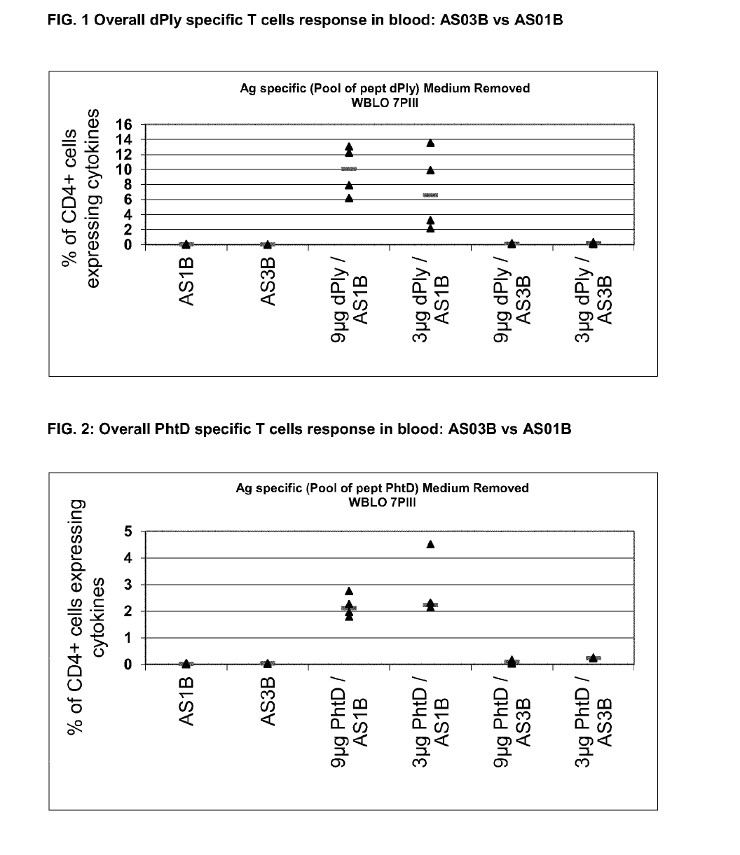

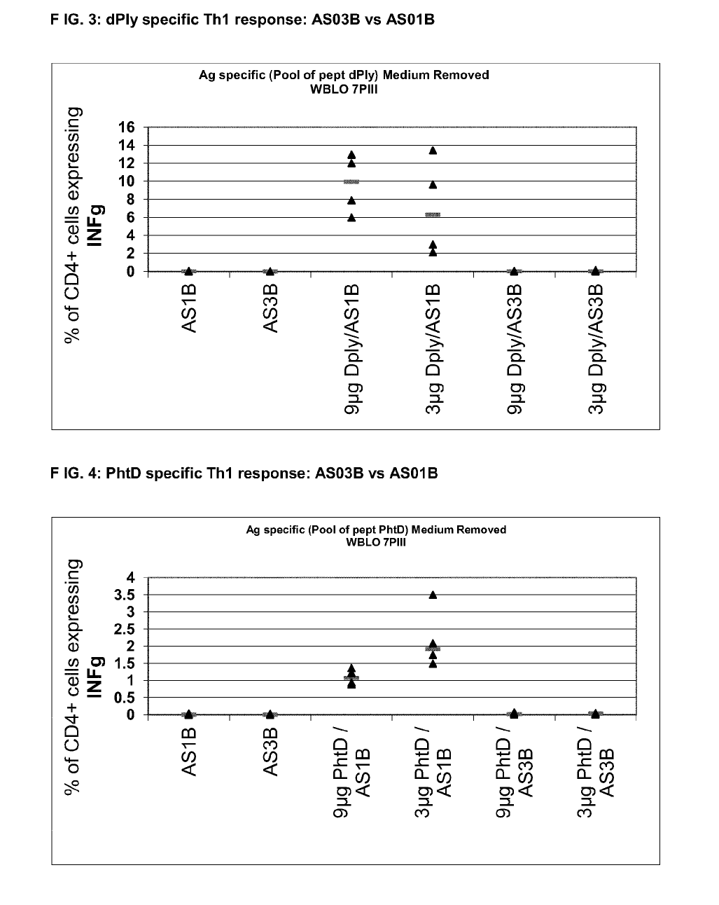

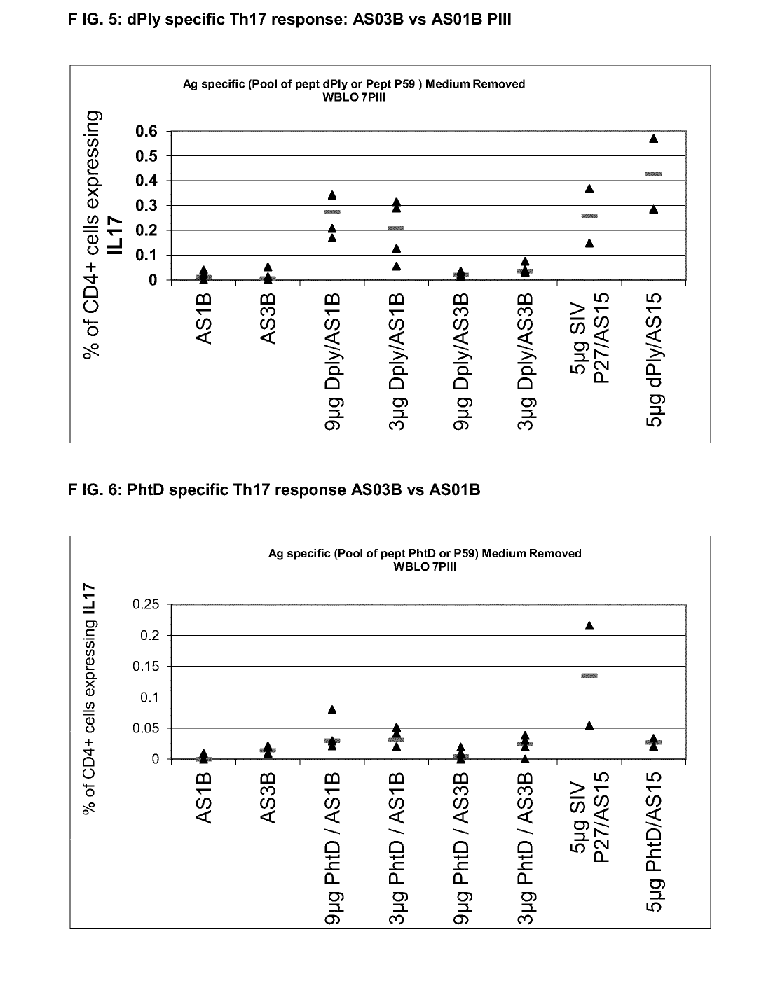

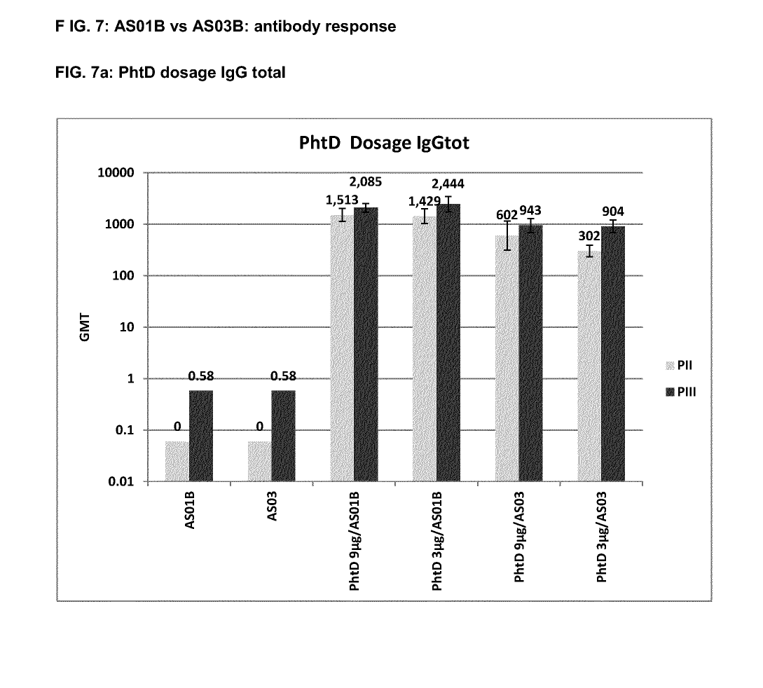

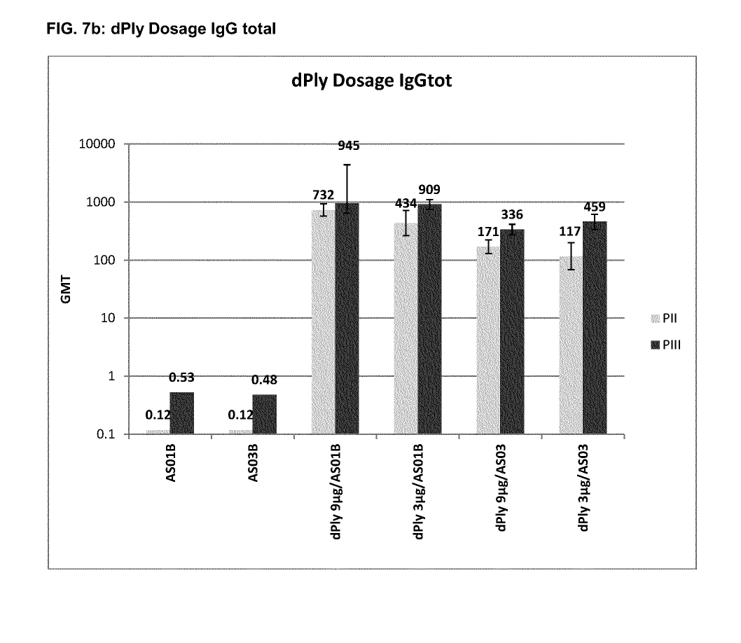

### DETAILED DESCRIPTION OF THE INVENTION

The present invention provides an immunogenic composition comprising at least one unconjugated Streptococcus pneumoniae protein selected from: pneumolysin and member(s) of the Polyhistidine Triad family (e.g. PhtD); and an adjuvant comprising QS21, monophosphoryl lipid A (MPL), phospholipid and sterol, presented in the form of a liposome. The S. pneumoniae protein is “unconjugated” which means that the protein is not covalently bound to a saccharide, e.g. as a carrier protein.

### Pneumolysin

In one aspect, the present invention provides an immunogenic composition comprising at least one unconjugated S. pneumoniae protein selected from: pneumolysin; and an adjuvant comprising QS21, monophosphoryl lipid A (MPL), phospholipid and sterol, presented in the form of a liposome. In an embodiment, immunogenic compositions of the invention comprise 3 to 90, 3 to 20, 20 to 40 or 40 to 70 μg (e.g. 10, 30 or 60 μg) unconjugated pneumococcal pneumolysin, per human dose.

By pneumolysin, or “Ply”, it is meant: native or wild-type pneumolysin from pneumococcus, recombinant pneumolysin, and fragments and/or variants thereof. In an embodiment, pneumolysin is native or wild-type pneumolysin from pneumococcus or recombinant pneumolysin. Pneumolysin is a 53 kDa thiol-activated cytolysin found in all strains of S. pneumoniae, which is released on autolysis and contributes to the pathogenesis of S. pneumoniae. It is highly conserved with only a few amino acid substitutions occurring between the Ply proteins of different serotypes. Pneumolysin is a multifunctional toxin with a distinct cytolytic (hemolytic) and complement activation activities (Rubins et al., Am. Respi. Cit Care Med, 153:1339-1346 (1996)). Its effects include for example, the stimulation of the production of inflammatory cytokines by human monocytes, the inhibition of the beating of cilia on human respiratory epithelial, and the decrease of bactericidal activity and migration of neutrophils. The most obvious effect of pneumolysin is in the lysis of red blood cells, which involves binding to cholesterol. Expression and cloning of wild-type or native pneumolysin is known in the art. See, for example, Walker et al. (Infect Immun, 55:1184-1189 (1987)), Mitchell et al. (Biochim Biophys Acta, 1007:67-72 (1989) and Mitchell et al (NAR, 18:4010 (1990)). WO2010/071986 describes wild-type Ply, e.g. SEQ IDs 2-42 (for example SEQ IDs 34, 35, 36, 37, 41). In one aspect, pneumolysin is Seq ID No. 34 of WO2010/071986. In another aspect, pneumolysin is Seq ID No. 35 of WO2010/071986. In another aspect, pneumolysin is Seq ID No. 36 of WO2010/071986. In another aspect, pneumolysin is Seq ID No. 37 of WO2010/071986. In another aspect, pneumolysin is Seq ID No. 41 of WO2010/071986. Furthermore, EP1601689B1 describes methods for purifying bacterial cytolysins such as pneumococcal pneumolysin by chromatography in the presence of detergent and high salt.

The term “fragment” as used in this specification is a moiety that is capable of eliciting a humoral and/or cellular immune response in a host animal. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40 or 50 contiguous amino acids of the full length sequence. Fragments may be readily modified by adding or removing 1, 2, 3, 4, 5, 6, 7, 8, 9, 10, 20, 30, 40 or 50 amino acids from either or both of the N and C termini.

The term “conservative amino acid substitution” as used in this specification involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deletion” as used in this specification is the removal of one or more amino acid residues from the protein sequence. Typically, no more than about from 1 to 6 residues (e.g. 1 to 4 residues) are deleted at any one site within the protein molecule.

The term “insertion” as used in this specification is the addition of one or more non-native amino acid residues in the protein sequence. Typically, no more than about from 1 to 6 residues (e.g. 1 to 4 residues) are inserted at any one site within the protein molecule.

In an embodiment, the present invention includes fragments and/or variants of pneumolysin, having differences in nucleic acid or amino acid sequences as compared to a wild type sequence. Where fragments of pneumolysin are used, these fragments will be at least about 15, at least about 20, at least about 40, or at least about 60 contiguous amino acid residues in length. In an embodiment of the invention, immunogenic fragments of pneumolysin comprise at least about 15, at least about 20, at least about 40, or at least about 60 contiguous amino acid residues of the full length sequence, wherein said polypeptide is capable of eliciting an immune response specific for said amino acid sequence. Pneumolysin is known to consist of four major structural domains (Rossjohn et al. Cell. 1997 May 30; 89(5):685-92). These domains may be modified by removing and/or modifying one or more of these domains. In an embodiment, the or each fragment contains exactly or at least 1, 2 or 3 domains. In another embodiment, the or each fragment contains exactly or at least 2 or 3 domains. In another embodiment, the or each fragment contains at least 3 domains. The or each fragment may be more than 50, 60, 70, 80, 90 or 100% identical to a wild type pneumolysin sequence.

In accordance with the present invention, a variant of pneumolysin includes sequences in which one or more amino acids are substituted and/or deleted and/or inserted compared to the wild type sequence. Amino acid substitution may be conservative or non-conservative. In one aspect, amino acid substitution is conservative. Substitutions, deletions, insertions or any combination thereof may be combined in a single variant so long as the variant is an immunogenic polypeptide. Variants of pneumolysin typically include any pneumolysin or any fragment of pneumolysin which shares at least 80, 90, 94, 95, 98, or 99% amino acid sequence identity with a wild-type pneumolysin sequence, e.g. SEQ IDs 2-42 from WO2010/071986 (for example SEQ IDs 34, 35, 36, 37, 41). In an embodiment, variants of pneumolysin typically include any pneumolysin or any fragment of pneumolysin which shares at least 80, 90, 94, 95, 98, or 99% amino acid sequence identity with SEQ ID 36 from WO2010/07198. In an embodiment, the present invention includes fragments and/or variants in which several, 5 to 10, 1 to 5, 1 to 3, 1 to 2 or 1 amino acids are substituted, deleted, or added in any combination. In another embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]). Variants of pneumolysin are described for example in WO04/43376, WO05/108580, WO05/076696, WO10/071986, WO10/109325 (SEQ IDs 44, 45 and 46) and WO10/140,119 (SEQ IDs 50 and 51). In an embodiment, the immunogenic composition of the invention comprises a variant of pneumolysin, for example, those described in WO05/108580, WO05/076696, WO10/071,986.

In an embodiment of the invention, pneumolysin and its fragments and/or variants thereof, have an amino acid sequence sharing at least 80, 85, 90, 95, 98, 99 or 100% identity with the wild type sequence for pneumolysin, e.g. SEQ IDs 34, 35, 36, 37, 41 from WO2010/071986. In another embodiment of the invention, pneumolysin and its fragments and/or variants thereof, comprise at least about 15, at least about 20, at least about 40, or at least about 60 contiguous amino acid residues of the wild type sequence for pneumolysin.

Pneumolysin is usually administered after being detoxified (i.e. rendered non-toxic to a human when provided at a dosage suitable for protection). As used herein, it is understood that the term “dPly” refers to detoxified pneumolysin suitable for medical use (i.e. non toxic). Pneumolysin may be detoxified chemically and/or genetically. Therefore, in an embodiment, immunogenic compositions of the invention comprise dPly.

Detoxification of pneumolysin can be conducted by chemical means, e.g. using a crosslinking agent, such as formaldehyde, glutaraldehyde and a cross-linking reagent containing an N-hydroxysuccinomido ester and/or a maleimide group (e.g. GMBS) or a combination of these. Such methods are well known in the art for various toxins, see for example EP1601689B1, WO04/081515, WO2006/032499. The pneumolysin used in chemical detoxification may be a native or recombinant protein or a protein that has been genetically engineered to reduce its toxicity (see below). Fusion proteins of pneumolysin or fragments and/or variants of pneumolysin may also be detoxified by chemical means. Therefore, in an embodiment, immunogenic compositions of the invention may comprise pneumolysin which has been chemically detoxified, e.g. by a formaldehyde treatment.

Pneumolysin can also be genetically detoxified. Thus, the invention encompasses pneumococcal proteins which may be, for example, mutated proteins. The term “mutated” is used herein to mean a molecule which has undergone deletion, addition or substitution of one or more amino acids (e.g. 1, 2, 3, 4, 5, 6, 7, 8, 9, 10, 11 or 12 amino acids), for example by using well known techniques for site directed mutagenesis or any other conventional method. In one embodiment, the molecule has undergone deletion or substitution of 1-15, suitable 10-15 amino acids. The mutated sequences may remove undesirable activities such as membrane permeation, cell lysis, and cytolytic activity against human erythrocytes and other cells, in order to reduce the toxicity, whilst retaining the ability to induce anti-pneumolysin protective and/or neutralizing antibodies following administration to a human. Fusion proteins of pneumolysin or fragments and/or variants of pneumolysin may also be detoxified by genetic means. Any of these modifications may be introduced using standard molecular biology and biochemical techniques. For example, as described above, a mutant pneumolysin protein may be altered so that it is biologically inactive whilst still maintaining its immunogenic epitopes, see, for example, WO90/06951, Berry et al. (Infect Immun, 67:981-985 (1999)) and WO99/03884. For example, a pneumolysin protein may be detoxified by three amino acid substitutions comprising T₆₅ to C, G₂₉₃ to C and C₂₄₈ to A. Another example of a genetically detoxified pneumolysin that can be used in the present invention is SEQ ID 9 from WO2011/075823. Thus, in a further embodiment, immunogenic compositions of the invention may comprise pneumolysin which has been genetically detoxified.

A combination of techniques may be used to detoxify pneumolysin. For example, immunogenic compositions of the invention may comprise pneumolysin which has been chemically and genetically detoxified.

### Polyhistidine Triad Family Protein

In another aspect, the present invention provides an immunogenic composition comprising at least one unconjugated S. pneumoniae protein selected from: member(s) of the Polyhistidine Triad family (e.g. PhtD); and an adjuvant comprising QS21, monophosphoryl lipid A (MPL), phospholipid and sterol, presented in the form of a liposome. In an embodiment, immunogenic compositions of the invention comprise 3 to 90, 3 to 20, 20 to 40 or 40 to 70 μg (e.g. 10, 30 or 60 μg) unconjugated S. pneumoniae protein selected from: member(s) of the Polyhistidine Triad family (e.g. PhtD), per human dose.

The Pht (Poly Histidine Triad, PhtX) family comprises proteins PhtA, PhtB, PhtD, and PhtE. The family is characterized by a lipidation sequence, two domains separated by a proline-rich region and several histidine triads, possibly involved in metal or nucleoside binding or enzymatic activity, (3 to 5) coiled-coil regions, a conserved N-terminus and a heterogeneous C terminus.

The term “member(s) of the Polyhistidine Triad family” include full length polyhistidine triad family (Pht) proteins, fragments or fusion proteins or immunologically functional equivalents thereof. These may be selected from PhtA, PhtB, PhtD or PhtE proteins having an amino acid sequence sharing at least 80, 85, 90, 95, 98, 99 or 100% identity with a sequence disclosed in WO00/37105 or WO00/39299. Where fragments of Pht proteins are used (separately or as part of a fusion protein), these fragments will be at least about 15, at least about 20, at least about 40, or at least about 60 contiguous amino acid residues in length, e.g from a Pht amino acid sequence in WO00/37105 or WO00/39299 wherein said polypeptide is capable of eliciting an immune response specific for said amino acid sequence in WO00/37105 or WO00/39299. In an embodiment, the or each fragment contains exactly or at least 2, 3, 4 or 5 histidine triad motifs (optionally, with native Pht sequence between the 2 or more triads, or intra-triad sequence that is more than 50, 60, 70, 80, 90 or 100% identical to a native pneumococcal intra-triad Pht sequence. In an embodiment, the or each fragment contains exactly or at least 2, 3 or 4 coiled coil regions. Fusion proteins may be composed of full length or fragments of 2, 3 or 4 of PhtA, PhtB, PhtD, PhtE, for example PhtA/B, PhtA/E, PhtB/A, PhtB/E, PhtE/A, PhtE/B, PhtA/D, PhtB/D, PhtD/A, PhtD/B, PhtD/E and PhtE/D, wherein the proteins are linked with the first mentioned at the N-terminus (see for example WO01/98334).

With regards to the PhtX proteins, PhtA disclosed in WO98/18930, is also referred to Sp36. It is a protein from the polyhistidine triad family and has the type II signal motif. PhtB is disclosed in WO00/37105, and is also referred to Sp036B. Another member of the PhtB family is the C3-Degrading Polypeptide, as disclosed in WO00/17370. This protein also is from the polyhistidine triad family and has the type II signal motif. An immunologically functional equivalent is the protein Sp42 disclosed in WO98/18930. A PhtB truncate (approximately 79 kD) is disclosed in WO99/15675 which is also considered a member of the PhtX family. PhtE is disclosed in WO00/30299 and is referred to as BVH-3.

In one embodiment, the S. pneumoniae protein selected from member(s) of the Polyhistidine Triad family is PhtD. The term “PhtD” as used herein includes the full length protein with the signal sequence attached or the mature full length protein with the signal peptide (for example 20 amino acids at N-terminus) removed, and fragments, variants and/or fusion proteins thereof, e.g. SEQ ID NO: 4 of WO00/37105. PhtD is also referred to “Sp036D”. In one aspect, PhtD is the full length protein with the signal sequence attached e.g. SEQ ID NO: 4 of WO00/37105. In another aspect, PhtD is a sequence comprising the mature full length protein with the signal peptide (for example 20 amino acids at N-terminus) removed, e.g. amino acids 21-838 of SEQ ID NO: 4 of WO00/37105. Suitably, the PhtD sequence comprises an N-terminal methionine. The present invention also includes PhtD polypeptides which are immunogenic fragments of PhtD, variants of PhtD and/or fusion proteins of PhtD. For example, as described in WO00/37105, WO00/39299, U.S. Pat. No. 6,699,703 and WO09/12588.

Where fragments of PhtD proteins are used (separately or as part of a fusion protein), these fragments will be at least about 15, at least about 20, at least about 40, or at least about 60 contiguous amino acid residues in length, e.g from a PhtD amino acid sequence in WO00/37105 or WO00/39299, such as SEQ ID NO: 4 of WO00/37105. In an embodiment of the invention, immunogenic fragments of PhtD protein comprise at least about 15, at least about 20, at least about 40, or at least about 60 contiguous amino acid residues of the sequence shown in SEQ ID NO: 4 of WO00/37105, wherein said polypeptide is capable of eliciting an immune response specific for said amino acid sequence. In an embodiment, the immunogenic composition of the invention comprises a fragment of PhtD, for example described in WO09/12601, WO01/98334 and WO09/12588. Where fragments of PhtD proteins are used (separately or as part of a fusion protein), each fragment optionally contains one or more histidine triad motif(s) of such polypeptides. A histidine triad motif is the portion of polypeptide that has the sequence HxxHxH where H is histidine and x is an amino acid other than histidine. In an embodiment of the present invention, the or each fragment contains exactly or at least 2, 3, 4 or 5 histidine triad motifs (optionally, with native PhtD sequence between the 2 or more triads, or intra-triad sequence) where the fragment is more than 50, 60, 70, 80, 90 or 100% identical to a native pneumococcal intra-triad PhtD sequence (e.g. the intra-triad sequence shown in SEQ ID NO: 4 of WO00/37105). Fragments of PhtD proteins optionally contain one or more coiled coil regions of such polypeptides. A coiled coil region is a region predicted by “Coils” algorithm Lupus, A et al (1991) Science 252; 1162-1164. In an embodiment of the present invention, the or each fragment contains exactly or at least 2, 3 or 4 coiled coil regions. In an embodiment of the present invention, the or each fragment contains exactly or at least 2, 3 or 4 coiled coil regions where the fragment is more than 50, 60, 70, 80, 90, 95, 96 or 100% identical to a native pneumococcal PhtD sequence (e.g. the sequence shown in SEQ ID NO: 4 of WO00/37105). In another embodiment of the present invention, the or each fragment includes one or more histidine triad motif as well as at least 1, 2, 3 or 4 coiled coil regions.

In the case where the PhtD polypeptide is a variant, the variation is generally in a portion thereof other than the histidine triad residues and the coiled-coil region, although variations in one or more of these regions may be made. In accordance with the present invention, a polypeptide variant includes sequences in which one or more amino acids are substituted and/or deleted and/or inserted compared to the wild type sequence. Amino acid substitution may be conservative or non-conservative. In one aspect, amino acid substitution is conservative. Substitutions, deletions, insertions or any combination thereof may be combined in a single variant so long as the variant is an immunogenic polypeptide. Variants of PhtD typically include any fragment or variation of PhtD which shares at least 80, 90, 95, 96, 98, or 99% amino acid sequence identity with a wild-type PhtD sequence, e.g. SEQ ID NO: 4 of WO00/37105. In an embodiment, the present invention includes fragments and/or variants in which several, 5 to 10, 1 to 5, 1 to 3, 1 to 2 or 1 amino acids are substituted, deleted, or added in any combination. In another embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]). Variants can be produced by conventional molecular biology techniques. Variants as used herein may also include naturally occurring PhtD alleles from alternate Streptococcus strains that exhibit polymorphisms at one or more sites within the homologous PhtD gene.

Fusion proteins are composed of full length or fragments of PhtD and PhtA, PhtB, and/or PhtE. Examples of fusion proteins are PhtA/D, PhtB/D, PhtD/A, PhtD/B, PhtD/E and PhtE/D, wherein the proteins are linked with the first mentioned at the N-terminus (see for example WO01/98334). The fusion fragment or fusion polypeptide may be produced, for example, by recombinant techniques or by the use of appropriate linkers for fusing previously prepared polypeptides or active fragments.

In an embodiment of the invention, PhtD and its fragments, variants and/or fusion proteins thereof comprise an amino acid sequence sharing at least 80, 85, 90, 95, 96, 97, 98, 99 or 100% identity with amino acid sequence 21 to 838 of SEQ ID NO:4 of WO00/37105. In another embodiment of the invention, PhtD and its fragments, variants and/or fusion proteins thereof have an amino acid sequence sharing at least 80, 85, 90, 95, 96, 97, 98, 99 or 100% identity with amino acid sequence 21 to 838 of SEQ ID NO:4 of WO00/37105. Suitably, PhtD and its fragments, variants and/or fusion proteins thereof comprise an amino acid sequence having an N-terminal methionine. In another embodiment of the invention, PhtD and its fragments, variants and/or fusion proteins thereof comprise at least about 15, at least about 20, at least about 40, or at least about 60 or at least about 100, or at least about 200, or at least about 400 or at least about 800 contiguous amino acid residues of the sequence shown in SEQ ID NO: 4 of WO00/37105.

In an embodiment of the invention, PhtD and its fragments, variants and/or fusion proteins thereof comprise an amino acid sequence sharing at least 80, 85, 90, 95, 96, 97, 98, 99 or 100% identity with amino acid sequence SEQ ID NO:73 of WO00/39299. In another embodiment of the invention, PhtD and its fragments, variants and/or fusion proteins thereof have an amino acid sequence sharing at least 80, 85, 90, 95, 96, 97, 98, 99 or 100% identity with amino acid sequence SEQ ID NO:73 of WO00/39299. In another embodiment of the invention, PhtD and its fragments, variants and/or fusion proteins thereof comprise at least about 15, at least about 20, at least about 40, or at least about 60, or at least about 100, or at least about 200, or at least about 400 or at least about 800 contiguous amino acid residues of the sequence shown in SEQ ID NO: 73 of WO00/39299. In another embodiment of the invention, the PhtD sequence is SEQ ID NO. 1 or 5 from WO2011/075823.

The present invention also includes PhtD proteins which differ from naturally occurring S. pneumoniae polypeptides in ways that do not involve the amino acid sequence. Non-sequence modifications include changes in acetylation, methylation, phosphorylation, carboxylation, or glycosylation. Also within the invention are those with modifications which increase peptide stability; such analogs may contain, for example, one or more non-peptide bonds (which replace the peptide bonds) in the peptide sequence. Also within the invention are analogs that include residues other than naturally occurring L-amino acids, e.g. D-amino acids or non-naturally occurring or synthetic amino acids, e.g. β or γ amino acids, and cyclic analogs.

In one aspect, immunogenic compositions of the invention comprise at least one unconjugated S. pneumoniae protein selected from: pneumolysin (e.g. dPly) and PhtD (e.g. a sequence comprising amino acids 21 to 838 of SEQ ID NO: 4 of WO00/37105); and an adjuvant comprising QS21, monophosphoryl lipid A (MPL), phospholipid and sterol, presented in the form of a liposome. Immunogenic compositions of the present invention may also contain two or more different unconjugated S. pneumoniae protein antigens. In another aspect, immunogenic compositions of the invention comprise 2 or more unconjugated S. pneumoniae proteins selected from: pneumolysin and PhtD. In another embodiment, immunogenic compositions of the invention comprise pneumolysin and PhtD. For example, immunogenic compositions of the invention may comprise unconjugated pneumolysin, e.g. dPly, and unconjugated pneumococcal PhtD.

### QS21

The present inventors have found that an immunogenic composition combining at least one unconjugated S. pneumoniae protein selected from: pneumolysin and member(s) of the Polyhistidine Triad family (e.g. PhtD); and an adjuvant comprising QS21 and monophosphoryl lipid A (MPL), provides advantageous properties.

QS-21 is a purified saponin fraction from the bark extracts of the South American tree Quillaja saponaria. QS21 typically comprises two principal isomers that share a triterpene, a branched trisaccharide, and a glycosylated pseudodimeric acyl chain. The two isomeric forms differ in the constitution of the terminal sugar within the linear tetrasaccharide segment, wherein the major isomer, QS-21-Api incorporates a β-D-apiose residue, and the minor isomer, QS-21-Xyl terminates in a β-D-xylose substituent. (Cleland, J. L. et al. J. Pharm. Sci. 1996, 85, 22-28).

QS21 may be prepared by HPLC purification from Quil A. Quil A was described as having adjuvant activity by Dalsgaard et al. in 1974 (“Saponin adjuvants”, Archiv. für die gesamte Virusforschung, Vol. 44, Springer Verlag, Berlin, p 243-254). Methods for production of QS21 are described in U.S. Pat. No. 5,057,540 (as QA21) and EP0362278. In an embodiment, immunogenic compositions of the invention contain QS21 in substantially pure form, that is to say, the QS21 is at least 90% pure, for example at least 95% pure, or at least 98% pure.

The dose of QS21 is suitably able to enhance an immune response to an antigen in a human. In particular a suitable QS21 amount is that which improves the immunological potential of the composition compared to the unadjuvanted composition, or compared to the composition adjuvanted with another QS21 amount, whilst being acceptable from a reactogenicity profile. QS21 can be used, for example, at an amount of 1 to 100 μg per composition dose, for example in an amount of 10 to 50 μg per composition dose. A suitable amount of QS21 is for example any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg per composition dose. In an embodiment, QS21 amount ranges from 25 to 75 μg per composition dose. In an embodiment, QS21 amount ranges from 1 to 30 μg per composition dose, suitably 5 to 20 μg per composition dose, for example 5 to 15 μg per composition dose, or 6 to 14 μg per composition dose, or 7 to 13 μg per composition dose. In an embodiment, a final concentration of 100 μg of QS21, is contained per ml of vaccine composition, or 50 μg per 0.5 ml vaccine dose. In another embodiment, a final concentration of 50 μg of QS21, is contained per ml of vaccine composition, or 25 μg per 0.5 ml vaccine dose. Specifically, a 0.5 ml vaccine dose volume contains 25 μg or 50 μg of QS21 per dose. In an embodiment, immunogenic compositions of the invention comprise 5 to 60, 45 to 55, or 20 to 30 μg (e.g. 20, 25, 30, 35, 40, 45 or 50 μg) of QS21. For example, immunogenic compositions of the invention may comprise 50 μg of QS21, per human dose. Suitably, the ratio of S. pneumoniae protein:QS21 is 0.05:1 to 3:1, e.g. 1:1 to 3:1 by weight (w/w) (μg).

### Monophosphoryl Lipid A

Monophosphoryl lipid A (MPL) is a nontoxic derivative of the lipopolysaccharide (LPS) of gram-negative bacteria, e.g. Salmonella minnesota R595. It retains adjuvant properties of the LPS while demonstrating a reduced toxicity (Johnson et al. 1987 Rev. Infect. Dis. 9 Suppl:S512-S516). MPL is composed of a series of 4′-monophosphoryl lipid A species that vary in the extent and position of fatty acid substitution. It may be prepared by treating LPS with mild acid and base hydrolysis followed by purification of the modified LPS. For example, LPS may be refluxed in mineral acid solutions of moderate strength (e.g. 0.1 M HCl) for a period of approximately 30 minutes. This process results in dephosphorylation at the 1 position, and decarbohydration at the 6′ position. The term “monophosphoryl lipid A (MPL)” as used herein includes derivatives of monophosphoryl lipid A. Derivatives of monophosphoryl lipid A include 3D-MPL and synthetic derivatives.

3D-MPL is 3-O-deacylated monophosphoryl lipid A (or 3 De-O-acylated monophosphoryl lipid A). Chemically it is a mixture of 3-deacylated monophosphoryl lipid A with 4, 5 or 6 acylated chains. 3D-MPL is available under the trademark MPL® by GlaxoSmithKline Biologicals North America. 3-O-deacylated monophosphoryl lipid A (3D-MPL). It has a further reduced toxicity while again maintaining adjuvanticity, and may typically be prepared by mild alkaline hydrolysis, see for example U.S. Pat. No. 4,912,094. Alkaline hydrolysis is typically performed in organic solvent, such as a mixture of chloroform/methanol, by saturation with an aqueous solution of weak base, such as 0.5 M sodium carbonate at pH 10.5. For further information on the preparation of 3D-MPL see GB2220211A and WO02078637 (Corixa Corporation). In one aspect of the present invention small particle 3 D-MPL may be used. Small particle 3D-MPL has a particle size such that it may be sterile-filtered through a 0.22 μm filter. Such preparations are described in International Patent Application No. WO94/21292. In an embodiment, immunogenic compositions of the invention comprise 3-O-Deacylated monophosphoryl lipid A (3D-MPL).

Lipopolysaccharide (LPS) from gram-negative bacteria and its derivatives, or fragments thereof, including 3D-MPL are TLR-4 (Toll-like receptor 4) ligands, capable of causing a signalling response through the TLR-4 signalling pathway (Sabroe et al, JI 2003 p 1630-5). Toll-like receptors (TLRs) are type I transmembrane receptors, evolutionarily conserved between insects and humans. Ten TLRs have so far been established (TLRs 1-10). Members of the TLR family have similar extracellular and intracellular domains; their extracellular domains have been shown to have leucine-rich repeating sequences, and their intracellular domains are similar to the intracellular region of the interleukin-1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NF-KB activation of cytokines; this Myd88 pathway is one way by which cytokine release is effected by TLR activation. Research carried out so far has found that TLRs recognise different types of agonists, although some agonists are common to several TLRs.

Synthetic derivatives of lipid A are known and thought to be TLR 4 agonists include, but are not limited to: OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO95/14026); OM 294 DP (3S,9R)-3-[(R)-dodecanoyloxytetradecanoylamino]-4-oxo-5-aza-9-(R)-[(R)-3-hydroxytetradecanoylamino]decan-1,10-diol,1,10-bis(dihydrogenophosphate) (WO99/64301 and WO00/0462); OM 197 MP-Ac DP (3S—,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO01/46127).

The dose of monophosphoryl lipid A (MPL), e.g. 3D-MPL, is suitably able to enhance an immune response to an antigen in a human. In particular a suitable monophosphoryl lipid A (MPL), e.g. 3D-MPL, amount is that which improves the immunological potential of the composition compared to the unadjuvanted composition, or compared to the composition adjuvanted with another MPL amount, whilst being acceptable from a reactogenicity profile. Monophosphoryl lipid A (MPL), e.g. 3D-MPL, can be used, for example, at an amount of 1 to 100 μg per composition dose, for example in an amount of 10 to 50 μg per composition dose. A suitable amount of monophosphoryl lipid A (MPL), e.g. 3D-MPL, is for example any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg per composition dose. In an embodiment, monophosphoryl lipid A (MPL), e.g. 3D-MPL, amount ranges from 25 to 75 μg per composition dose. In an embodiment, 3D-MPL amount ranges from 1 to 30 μg per composition dose, suitably 5 to 20 μg per composition dose, for example 5 to 15 μg per composition dose, or 6 to 14 μg per composition dose, or 7 to 13 μg per composition dose. In an embodiment, a final concentration of 100 μg of monophosphoryl lipid A (MPL), e.g. 3D-MPL, is contained per ml of vaccine composition, or 50 μg per 0.5 ml vaccine dose. In another embodiment, a final concentration of 50 μg of monophosphoryl lipid A (MPL), e.g. 3D-MPL, is contained per ml of vaccine composition, or 25 μg per 0.5 ml vaccine dose. Specifically, a 0.5 ml vaccine dose volume contains 25 μg or 50 μg of monophosphoryl lipid A (MPL), e.g. 3D-MPL, per dose. In one aspect, immunogenic compositions of the invention comprise 5 to 60, 45 to 55, or 20 to 30 μg (e.g. 20, 25, 30, 35, 40, 45 or 50 μg) monophosphoryl lipid A (MPL). For example, immunogenic compositions of the invention may comprise 50 μg of 3D-MPL, per human dose. Suitably, the ratio of the ratio of S. pneumoniae protein:monophosphoryl lipid A (MPL), e.g. 3D-MPL, is 0.05:1 to 3:1, e.g. 1:1 to 3:1 by weight (w/w) (μg).

In another embodiment, other natural or synthetic agonists of TLR molecules are used as optional additional immunostimulants. These could include, but are not limited to agonists for TLR1, TLR2, TLR3, TLR4, TLR5, TLR6, TLR7, TLR8 and TLR9 or a combination thereof (for examples see Sabroe et al, JI 2003 p 1630-5). Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants. Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, muramyl dipeptide (MDP) or F protein of respiratory syncitial virus. In an embodiment the TLR agonist is HSP 60, 70 or 90.

In an embodiment of the invention, QS21 and monophosphoryl lipid A (MPL), e.g. 3D-MPL, are present in the same final concentration per human dose of the immunogenic composition. In another embodiment, a human dose of the immunogenic composition of the invention comprises a final level of 50 μg of monophosphoryl lipid A (MPL), e.g. 3D-MPL, and 50 μg of QS21. In a further embodiment, a human dose of the immunogenic composition of the invention comprises a final level of 25 μg of monophosphoryl lipid A (MPL), e.g. 3D-MPL, and 25 μg of QS21.

### Liposome Carrier

The adjuvant used for the compositions of the invention comprises a liposome carrier. Liposomes may be made from phospholipids (such as dioleoyl phosphatidyl choline, DOPC) and sterol, e.g. cholesterol, using techniques known in the art. Such liposome carriers may carry the QS21 and/or monophosphoryl lipid A (MPL), e.g. 3D-MPL. Suitable compositions of the invention are those wherein liposomes are initially prepared without MPL (as described in WO96/33739), and MPL is then added, suitably as small particles of below 100 nm particles or particles that are susceptible to sterile filtration through a 0.22 μm membrane. The MPL is therefore not contained within the vesicle membrane (known as MPL out). Compositions where the MPL is contained within the vesicle membrane (known as MPL in) also form an aspect of the invention. The unconjugated S. pneumoniae proteins can be contained within the vesicle membrane or contained outside the vesicle membrane. Suitably soluble antigens are outside and hydrophobic or lipidated antigens are either contained inside or outside the membrane. Encapsulation within liposomes is described in U.S. Pat. No. 4,235,877.

The liposomes of the present invention comprise a phospholipid, for example a phosphatidylcholine, which may be non-crystalline at room temperature, for example eggyolk phosphatidylcholine, dioleoyl phosphatidylcholine or dilauryl phosphatidylcholine. Suitably, the phospholipid is dioleoylphosphatidylcholine (DOPC). A further aspect is an immunogenic composition of the invention comprising 0.1 to 10 mg, 0.2 to 7, 0.3 to 5, 0.4 to 2, or 0.5 to 1 mg (e.g. 0.4 to 0.6, 0.9 to 1.1, 0.5 or 1 mg) phospholipid. In one particular embodiment of the invention, the amount of DOPC is 1000 μg, per human dose. In another particular embodiment of the invention, the amount of DOPC is 500 μg, per human dose.

The liposomes of the present invention comprise a sterol. The sterol increases the stability of the liposome structure. Suitable sterols include β-sitosterol, stigmasterol, ergosterol, ergocalciferol and cholesterol. These sterols are well known in the art, for example cholesterol is disclosed in the Merck Index, 11th Edn., page 341, as a naturally occurring sterol found in animal fat. In one particular embodiment of the invention, the sterol is cholesterol. Typically, the sterol may be added during formulation of the antigen preparation using QS21 quenched with the sterol as described in WO96/33739.

The amount of sterol to phospholipid is 1 to 50% (w/w), suitably 20 to 35%, e.g. 25%. The ratio of QS21:sterol is suitably between 1:10 to 1:1 (w/w), Suitably excess sterol is present, the ratio of QS21:sterol being at least 1:2 (w/w), for example 1:5 (w/w). In an embodiment, the immunogenic compositions of the invention comprise 0.025 to 2.5, 0.05 to 1.5, 0.075 to 0.75, 0.1 to 0.3, or 0.125 to 0.25 mg (e.g. 0.2 to 0.3, 0.1 to 0.15, 0.25 or 0.125 mg) sterol. In a further embodiment, immunogenic compositions of the invention comprise 250 μg of sterol, e.g. cholesterol, per human dose. In a further embodiment, immunogenic compositions of the invention comprise 125 μg of sterol, e.g. cholesterol, per human dose.

Liposomes of the invention will suitably be comprised in a liquid medium. The liquid medium comprises physiologically acceptable liquids such as water, aqueous salt solutions and buffer solutions, e.g PBS etc. For example, immunogenic compositions of the invention may comprise water and sodium phosphate buffer.

In one aspect of the invention, the adjuvant is AS01B (see e.g. WO96/33739). In another aspect of the invention, the adjuvant is AS01E (see e.g. WO2007/068907).

### Additional Antigens

The immunogenic compositions of the present invention may comprise additional antigens capable of eliciting an immune response against a human or animal pathogen. These additional antigens include for example additional S. pneumoniae antigens, e.g. S. pneumoniae protein antigens. Where the additional antigen is a pneumococcal protein, the protein is optionally conjugated for example to a saccharide. Optionally, the pneumococcal protein is unconjugated or present in the immunogenic composition as a free protein.

In an embodiment, the immunogenic compositions of the invention comprise at least 1 additional protein selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), PspA, PsaA, Sp128, Sp101, Sp130, Sp125 and Sp133. In a further embodiment, the immunogenic compositions of the invention comprise two or more additional proteins selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), PspA, PsaA, and Sp128. In a further embodiment, the immunogenic compositions of the invention comprises two or more additional proteins selected from the group consisting of the Poly Histidine Triad family (PhtX), Choline Binding Protein family (CbpX), CbpX truncates, LytX family, LytX truncates, CbpX truncate-LytX truncate chimeric proteins (or fusions), and Sp128.

Concerning the Choline Binding Protein family (CbpX), members of that family comprise an N terminal region (N), conserved repeat regions (R1 and/or R2), a proline rich region (P) and a conserved choline binding region (C), made up of multiple repeats, that comprises approximately one half of the protein. As used in this application, the term “Choline Binding Protein family (CbpX)” is selected from the group consisting of Choline Binding Proteins as identified in WO97/41151, PbcA, SpsA, PspC, CbpA, CbpD, and CbpG. CbpA is disclosed in WO97/41151. CbpD and CbpG are disclosed in WO00/29434. PspC is disclosed in WO97/09994. PbcA is disclosed in WO98/21337. SpsA is a Choline binding protein disclosed in WO98/39450. Optionally the Choline Binding Proteins are selected from the group consisting of CbpA, PbcA, SpsA and PspC.

An embodiment of the invention comprises CbpX truncates wherein “CbpX” is defined above and “truncates” refers to CbpX proteins lacking 50% or more of the Choline binding region (C). Optionally such proteins lack the entire choline binding region. Optionally, the such protein truncates lack (i) the choline binding region and (ii) a portion of the N-terminal half of the protein as well, yet retain at least one repeat region (R1 or R2). Optionally, the truncate has 2 repeat regions (R1 and R2). Examples of such embodiments are NR1xR2 and R1xR2 as illustrated in WO99/51266 or WO99/51188, however, other choline binding proteins lacking a similar choline binding region are also contemplated within the scope of this invention. In another embodiment, immunogenic compositions of the invention may comprise an immunogenic polypeptide of PcpA, for example selected from S. pneumoniae TIGR4, S. pneumoniae 14453, S. pneumoniae B6 (GenBank Accession No. CAB04758), or S. pneumoniae R6 (GenBank Accession No. NP_{—}359536). In one embodiment, the immunogenic polypeptide PcpA lacks the N-terminal signal sequence. In another embodiment, the immunogenic polypeptide PcpA lacks the choline binding domain anchor sequence that is found in the naturally occurring sequence. In another embodiment, the immunogenic polypeptide PcpA lacks bother the signal sequence and the choline binding domain(s). For example, immunogenic compositions of the invention may comprise an immunogenic polypeptide of PcpA having at least 50, 60, 70, 80, 90, 95, 97, 99% identity with SEQ ID No. 2 from WO2011/075823. In another embodiment, immunogenic compositions of the invention may comprise an immunogenic polypeptide of PcpA having the sequence SEQ ID No. 7 from WO2011/075823.

The LytX family is membrane associated proteins associated with cell lysis. The N-terminal domain comprises choline binding domain(s), however the LytX family does not have all the features found in the CbpA family noted above and thus for the present invention, the LytX family is considered distinct from the CbpX family. In contrast with the CbpX family, the C-terminal domain contains the catalytic domain of the LytX protein family. The family comprises LytA, B and C. With regards to the LytX family, LytA is disclosed in Ronda et al., Eur J Biochem, 164:621-624 (1987). LytB is disclosed in WO98/18930, and is also referred to as Sp46. LytC is also disclosed in WO98/18930, and is also referred to as Sp91. An embodiment of the invention comprises LytC.

Another embodiment comprises LytX truncates wherein “LytX” is defined above and “truncates” refers to LytX proteins lacking 50% or more of the Choline binding region. Optionally such proteins lack the entire choline binding region. Yet another embodiment of this invention comprises CbpX truncate-LytX truncate chimeric proteins (or fusions). Optionally this comprises NR1xR2 (or R1xR2) of CbpX and the C-terminal portion (Cterm, i.e., lacking the choline binding domains) of LytX (e.g. LytCCterm or Sp91Cterm). Optionally CbpX is selected from the group consisting of CbpA, PbcA, SpsA and PspC. Optionally, it is CbpA. Optionally, LytX is LytC (also referred to as Sp91). Another embodiment of the present invention is a PspA or PsaA truncate lacking the choline binding domain (C) and expressed as a fusion protein with LytX. Optionally, LytX is LytC.

With regards to PsaA and PspA, both are known in the art. For example, PsaA and transmembrane deletion variants thereof have been described by Berry & Paton, Infect Immun 1996 December; 64(12):5255-62. PspA and transmembrane deletion variants thereof have been disclosed in, for example, U.S. Pat. No. 5,804,193, WO92/14488, and WO99/53940.

Sp128 and Sp130 are disclosed in WO00/76540. Sp125 is an example of a pneumococcal surface protein with the Cell Wall Anchored motif of LPXTG (where X is any amino acid). Any protein within this class of pneumococcal surface protein with this motif has been found to be useful within the context of this invention, and is therefore considered a further protein of the invention. Sp125 itself is disclosed in WO98/18930, and is also known as ZmpB—a zinc metalloproteinase. Sp101 is disclosed in WO98/06734 (where it has the reference # y85993). It is characterized by a Type I signal sequence. Sp133 is disclosed in WO98/06734 (where it has the reference # y85992). It is also characterized by a Type I signal sequence.

The immunogenic compositions of the invention may also comprise S. pneumoniae capsular saccharides (suitably conjugated to a carrier protein). The saccharides (e.g. polysaccharides) may be derived from serotypes of pneumococcus such as serotypes 1, 2, 3, 4, 5, 6A, 6B, 7F, 8, 9N, 9V, 10A, 11A, 12F, 14, 15B, 17F, 18C, 19A, 19F, 20, 22F, 23F and 33F. In an embodiment, at least four serotypes are included in the composition, e.g. 6B, 14, 19F and 23F. In another embodiment, at least 7 serotypes are included in the composition, e.g. 4, 6B, 9V, 14, 18C, 19F and 23F. Suitably, each of the saccharides is conjugated to a carrier protein. In an embodiment, the immunogenic compositions of the invention comprise pneumolysin and/or member(s) of the Polyhistidine Triad family (e.g. PhtD) as carrier proteins.

### Dosage

The term “human dose” as used herein means a dose which is in a volume suitable for human use. Generally the final dose volume (vaccine composition volume) may be between 0.25 to 1.5 ml, 0.4 to 1.5 ml, or 0.4 to 0.6 ml. In an embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml. In another embodiment, in particular when the immunogenic composition is for the paediatric population, a human dose may be less than 0.5 ml such as between 0.25 and 0.5 ml.

The amount of S. pneumoniae protein in each dose is selected as an amount which induces an immunoprotective response without significant, adverse side effects in typical vaccines. Such amount will vary depending upon which specific immunogen is employed and how it is presented. Generally, it is expected that each dose will comprise 1-1000 μg of protein antigen, for example 1 to 500 μg, 1 to 100 μg, or 1 to 50 μg. An optimal amount for a particular immunogenic composition can be ascertained by standard studies involving observation of appropriate immune responses in subjects.

### Vaccination

The present invention provides a vaccine comprising the immunogenic compositions of the invention. Embodiments herein relating to “immunogenic compositions” of the invention are also applicable to embodiments relating to “vaccines” of the invention, and vice versa. In an embodiment, the vaccine comprises the immunogenic composition of the invention and a pharmaceutically acceptable excipient.

The vaccines of the invention may be administered by any suitable delivery route, such as intradermal, mucosal e.g. intranasal, oral, intramuscular or subcutaneous. Other delivery routes are well known in the art. Vaccine preparation is generally described in Vaccine Design (“The subunit and adjuvant approach” (eds Powell M. F. & Newman M. J.) (1995) Plenum Press New York).

In one aspect, the immunogenic composition of the invention is administered by the intramuscular delivery route. Intramuscular administration may be to the thigh or the upper arm. Injection is typically via a needle (e.g. a hypodermic needle), but needle-free injection may alternatively be used. A typical intramuscular dose is 0.5 ml.

Intradermal administration of the vaccine forms an embodiment of the present invention. Human skin comprises an outer “horny” cuticle, called the stratum corneum, which overlays the epidermis. Underneath this epidermis is a layer called the dermis, which in turn overlays the subcutaneous tissue. The conventional technique of intradermal injection, the “mantoux procedure”, comprises steps of cleaning the skin, and then stretching with one hand, and with the bevel of a narrow gauge needle (26 to 31 gauge) facing upwards the needle is inserted at an angle of between 10 to 15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO99/34850 and EP1092444, also the jet injection devices described for example in WO01/13977, U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO97/37705 and WO97/13537. Alternative methods of intradermal administration of the vaccine preparations may include conventional syringes and needles, or devices designed for ballistic delivery of solid vaccines (WO99/27961), or transdermal patches (WO97/48440, WO98/28037), or applied to the surface of the skin (transdermal or transcutaneous delivery WO98/20734, WO98/28037).

When the vaccines of the present invention are to be administered to the skin, or more specifically into the dermis, the vaccine is in a low liquid volume, particularly a volume of between about 0.05 ml and 0.2 ml.

Another suitable administration route is the subcutaneous route. Any suitable device may be used for subcutaneous delivery, for example classical needle. In one aspect of the invention, a needle-free jet injector service is used, such as that published in WO01/05453, WO01/05452, WO01/05451, WO01/32243, WO01/41840, WO01/41839, WO01/47585, WO01/56637, WO01/58512, WO01/64269, WO01/78810, WO01/91835, WO01/97884, WO02/09796, WO02/34317. In another aspect of the invention, the device is pre-filled with the liquid vaccine formulation.

Alternatively the vaccine is administered intranasally. Typically, the vaccine is administered locally to the nasopharyngeal area, e.g. without being inhaled into the lungs. It is desirable to use an intranasal delivery device which delivers the vaccine formulation to the nasopharyngeal area, without or substantially without it entering the lungs. Preferred devices for intranasal administration of the vaccines according to the invention are spray devices. Suitable commercially available nasal spray devices include Accuspray™ (Becton Dickinson).

In an embodiment, spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO91/13281 and EP311 863 and EP516636, incorporated herein by reference. Such devices are commercially available from Pfeiffer GmbH and are also described in Bommer, R. Pharmaceutical Technology Europe, September 1999.

In another embodiment, intranasal devices produce droplets (measured using water as the liquid) in the range 1 to 200 μm, e.g. 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is another embodiment of an intranasal delivery system for use with the vaccines according to the invention. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device may be used for administering the vaccines according to the invention.

A further aspect of the invention is a method of making a vaccine of the invention comprising the steps of mixing the unconjugated S. pneumoniae protein with the adjuvant composition.

Although the vaccine of the invention may be administered as a single dose, components thereof may also be co-administered together at the same time or at different times (for instance pneumococcal saccharide conjugates could be administered separately, at the same time or 1 to 2 weeks after the administration of the any bacterial protein component of the vaccine for optimal coordination of the immune responses with respect to each other). Following an initial vaccination, subjects may receive one or several booster immunisation adequately spaced.

In one aspect of the invention, the target population is a population which is unprimed, either being naive or having failed to respond previously to infection or vaccination. In another aspect, the target population is elderly persons suitably aged 65 years and over, younger high-risk adults (i.e. between 18 and 64 years of age) such as people working in health institutions, or those young adults with a risk factor such as cardiovascular and pulmonary disease, or diabetes. Another target population is all children 6 months of age and over, especially children 6 to 23 months of age. Another target population immunocompromised persons.

Immunogenic compositions of the present invention maybe used for both prophylatic and therapeutic purposes. Diseases caused by S. pneumoniae infections include pneumonia, acute sinusitis, otitis media, meningitis, bacteremia, septicemia, osteomyelitis, septic arthritis, endocarditis, peritonitis, pericarditis, cellulitis, and brain abscess. In an embodiment of the present invention, S. pneumoniae infections include pneumonia, otitis media, meningitis and bacteremia. In one embodiment, the disease caused by S. pneumoniae is pneumonia e.g. community-acquired pneumonia. In another embodiment, the disease caused by S. pneumoniae is Invasive pneumococcal disease (IPD), i.e. an infection in which S. pneumoniae may be isolated from the blood or another normally sterile site. In another embodiment, disease caused by S. pneumoniae is pneumonia, e.g. severe pneumonia. The condition known as “severe pneumonia” is characterized according to guidelines set forth by various organizations, including the American Thoracic Society (ATS) (Am J Respir Crit Care Med 2001; 163:1730-1754). For example, the ATS requires at least one major criterion, such as a need for mechanical ventilation or septic shock, in addition to other criteria for a diagnosis of severe pneumonia. Generally, severe pneumonia can result from acute lung disease, lung inflammatory disease, or any perturbations in lung function due to factors such as inflammation or coagulation. Immunogenic compositions of the present invention may also be useful in the treatment or prevention of AECOPD. In one aspect, immunogenic compositions of the present invention may be used in the treatment or prevention of AECOPD caused by Streptococcus pneumoniae.

Further aspects of the present invention include:
a method of eliciting an immune response by immunising a mammal with immunogenic compositions of the invention;
a method of treating or preventing a disease caused by Streptococcus pneumoniae infection comprising intramuscularly administering to a subject, e.g. human, in need thereof comprising administering to said subject, e.g. human, an immunogenic composition of the invention;
a method of treating or preventing a disease caused by S. pneumoniae infection comprising intramuscularly administering to a patient suffering from or susceptible to S. pneumoniae infection an immunogenic composition of the invention;
an immunogenic composition of the invention for use in treating or preventing a disease caused by S. pneumoniae infection;
use of an immunogenic composition of the invention in the manufacture of a medicament for use in treating or preventing a disease caused by S. pneumoniae infection;
use of an immunogenic composition of the invention in the manufacture of an intramuscular vaccine for use in treating or preventing a disease caused by S. pneumoniae infection.

### Immunogenic Properties

A further aspect of the invention is an immunogenic composition of the invention capable of invoking a T cell response in a mammal. In one aspect, the T cell response may be a cytolytic T cell response. Cytolytic T cell responses may be measured using standard assays for example by measuring the cytotoxic activity of T cells using a chromium release assay, e.g. ⁵¹Cr is added to target cells and the amount of ⁵¹Cr released by lysed cells is measured, or the expression of molecules involved in T cell cytotoxicity (e.g. granzymeB, perforin) by flow cytometry.

In one aspect, immunogenic compositions of the invention are capable of inducing an improved CD4 T-cell immune response against at least one of the component antigen(s) or antigenic composition compared to the CD4 T-cell immune response obtained with the corresponding composition which in un-adjuvanted, i.e. does not contain any exogeneous adjuvant (herein also referred to as ‘plain composition’) and/or other adjuvanted compositions known in the art.

By “improved CD4 T-cell immune response” is meant that a higher CD4 response is obtained in a mammal, e.g. human, after administration of the adjuvanted immunogenic composition than that obtained after administration of the same composition without adjuvant and/or with other known adjuvants. For example, a higher CD4 T-cell response is obtained in a mammal upon administration of an immunogenic composition of the invention, compared to the response induced after administration of an immunogenic composition which is un-adjuvanted and/or other adjuvanted compositions known in the art.

The improved CD4 T-cell immune response may be assessed by measuring the number of cells producing any of the following cytokines:
cells producing any cytokines (IFNγ, IL-2, IL-17, IL-13)
cells producing IFNγ
cells producing IL-17

There will be improved CD4 T-cell immune response when cells producing any of the above cytokines will be in a higher amount following administration of the immunogenic composition of the invention compared to the administration of the un-adjuvanted composition and/or other adjuvanted compositions. In an embodiment, at least one of the three conditions mentioned herein above will be fulfilled. In another embodiment, at least two of the three conditions mentioned herein above will be fulfilled. In another embodiment, all three of the conditions mentioned herein above will be fulfilled. In a further aspect, the immunogenic composition of the invention is capable of stimulating IFNγ production. IFNγ production may be measured as described in the Examples herein.

For example, IFNγ production may be measured by restimulating peripheral blood antigen specific CD4 and CD8 T cells in vitro using antigen corresponding to IFNγ, e.g. PhtD and dPly, conventional immunofluorescence labelling and measurement by flow cytometry to determine the frequency of cytokines positive CD4 or CD8 T cell within CD4 or CD8 cell sub-population. In a further aspect, the immunogenic composition of the invention is capable of stimulating IL-17 production. IL-17 production may be measured as described in the Examples herein. For example, IL-17 production may be measured by restimulating peripheral blood antigen specific CD4 and CD8 T cells in vitro using antigen corresponding to IL-17, e.g. PhtD and dPly, conventional immunofluorescence labelling and measurement by flow cytometry to determine the frequency of cytokines positive CD4 or CD8 T cell within CD4 or CD8 cell sub-population.

The invention will be further described by reference to the following, non-limiting, examples:

### Example 1

### Preclinical Comparison of AS01B vs AS03B Th Response in Mice Model (C57Bl6) for PhtD and dPly

Six weeks old C57bl6 mice were immunized by the IM route at days 0, 14 and 28 with 9 μg or 3 μg of PhtD or dPly formulated in AS01B or AS03B. Control groups were immunized with 5 μg of PhtD, dPly or Sivp27 (Sivp27 was used as a positive control) formulated in AS15. FACS analysis was performed 7 days after the second and the third immunizations on whole blood and nine days after the third immunizations on the spleen.

### Experiment 1:

| Group | Antigen/Formulation | Antigen dose |
|---|---|---|
| 1 | AS01B | |
| 2 | AS03B | |
| 3 | dPly/AS01B | 9 μg |
| 4 | dPly/AS01B | 3 μg |
| 5 | dPly/AS03B | 9 μg |
| 6 | dPly/AS03B | 3 μg |
| 7 | AS15/dPly | 5 μg |
| 8 | AS15/sivP17 (Th17 control) | 5 μg |

### Experiment 2:

| Group | Antigen/Formulation | Antigen dose |
|---|---|---|
| 1 | AS01B | |
| 2 | AS03B | |
| 3 | PhtD/AS01B | 9 μg |
| 4 | PhtD/AS01B | 3 μg |
| 5 | PhtD/AS03B | 9 μg |
| 6 | PhtD/AS03B | 3 μg |
| 7 | AS15/PhtD | 5 μg |
| 8 | AS15/sivP27 (Th17 control) | 5 μg |

### Preparation of the Adjuvant Formulations

### Final Composition of AS01B/Dose:

Liposomes: DOPC 1000 ug, cholesterol 250 ug, 3D-MPL 50 ug

### QS21 50 ug

PBS to volume 0.5 ml

### Final Composition of AS01E/Dose:

Liposomes: DOPC 500 ug, cholesterol 125 ug, 3D-MPL 25 ug

### QS21 25 ug

PBS to volume 0.5 ml

### Final Composition of AS03B/Dose:

Oil in water emulsion: squalene and DL-alpha-tocopherol

### Polysorbate 80 (Tween 80)

### Final Composition of AS15/Dose:

Liposomes: DOPC 1000 μg, cholesterol 250 μg, 3D-MPL 50 μg

### QS21 50 μg

### CpG7909: 420 μg

### Preparation of MPL/QS21 Liposomal Adjuvants, AS01:

The adjuvants, named AS01, comprises 3D-MPL and QS21 in a quenched form with cholesterol, and was made as described in WO 96/33739, incorporated herein by reference. In particular the AS01 adjuvant was prepared essentially as Example 1.1 of WO 96/33739. The AS01B adjuvant comprises: liposomes, which in turn comprise dioleoyl phosphatidylcholine (DOPC), cholesterol and 3D MPL [in an amount of 1000 μg DOPC, 250 μg cholesterol and 50 μg 3D-MPL, each value given approximately per vaccine dose], QS21 [50 μg/dose], phosphate NaCl buffer and water to a volume of 0.5 ml.

The AS01E adjuvant comprises the same ingredients than AS01B but at a lower concentration in an amount of 500 μg DOPC, 125 μg cholesterol, 25 μg 3D-MPL and 25 μg QS21, phosphate NaCl buffer and water to a volume of 0.5 ml.

In the process of production of liposomes containing MPL the DOPC (Dioleyl phosphatidylcholine), cholesterol and MPL are dissolved in ethanol. A lipid film is formed by solvent evaporation under vacuum. Phosphate Buffer Saline (9 mM Na₂HPO₄, 4 1 mM KH₂PO₄, 100 mM NaCl) at pH 6.1 is added and the mixture is submitted to prehomogenization followed by high pressure homogenisation at 15,000 psi (around 15 to 20 cycles). This leads to the production of liposomes which are sterile filtered through a 0.22 μm membrane in an aseptic (class 100) area. The sterile product is then distributed in sterile glass containers and stored in a cold room (+2 to +8° C.).

In this way the liposomes produced contain MPL in the membrane (the “MPL in” embodiment of WO 96/33739).

QS21 is added in aqueous solution to the desired concentration.

### Preparation of the Oil in Water Emulsion and Adjuvant Formulations AS03B:

Unless otherwise stated, the oil/water emulsion used in the subsequent examples is composed an organic phase made of 2 oils (alpha-tocopherol and squalene), and an aqueous phase of PBS containing Tween 80 as emulsifying agent. Unless otherwise stated, the oil in water emulsion adjuvant formulations used in the subsequent examples were made comprising the following oil in water emulsion component (final concentrations given): 2.5% squalene (v/v), 2.5% alpha-tocopherol (v/v), 0.9% polyoxyethylene sorbitan monooleate (v/v) (Tween 80), see WO 95/17210. This emulsion, termed AS03 in the subsequent examples, was prepared as followed as a two-fold concentrate.

### Preparation of Emulsion SB62:

The preparation of the SB62 emulsion is made by mixing under strong agitation of an oil phase composed of hydrophobic components (DL-α-tocopherol and squalene) and an aqueous phase containing the water soluble components (the anionic detergent Tween 80 and PBS mod (modified), pH 6.8). While stirring, the oil phase ( 1/10 total volume) is transferred to the aqueous phase ( 9/10 total volume), and the mixture is stirred for 15 minutes at room temperature. The resulting mixture then subjected to shear, impact and cavitation forces in the interaction chamber of a microfluidizer (15000 PSI—8 cycles, or 3 cycles in the adjuvant used in the clinical trial reported in Example III) to produce submicron droplets (distribution between 100 and 200 nm). The resulting pH is between 6.8±0.1. The SB62 emulsion is then sterilised by filtration through a 0.22 μm membrane and the sterile bulk emulsion is stored refrigerated in Cupac containers at 2 to 8° C. Sterile inert gas (nitrogen or argon) is flushed into the dead volume of the SB62 emulsion final bulk container for at least 15 seconds.

The final composition of the SB62 emulsion is as follows: Tween 80:1.8% (v/v) 19.4 mg/ml; Squalene: 5% (v/v) 42.8 mg/ml; α-tocopherol: 5% (v/v) 47.5 mg/ml; PBS-mod: NaCl 121 mM, KCl 2.38 mM, Na₂HPO₄ 7.14 mM, KH₂PO₄ 1.3 mM; pH 6.8±0.1.

### Preparation of the Adjuvant Formulations AS15:

The adjuvant system AS15 has been previously described WO 00/62800.

AS15 is a combination of the two adjuvant systems, AS01B the first is composed of liposomes containing 3D-MPL and QS21 and the second is composed of CpG 7909 (also known as CpG 2006) in phosphate buffer saline.

### Preparation of the Antigens

Preparation of dPly:

Pneumococcal pneumolysin was prepared and detoxified as described in WO2004/081515 and WO2006/32499 using formaldehyde detoxification.

### Expression and Purification of PhtD:

### Expression OF PhtD:

The PhtD protein is a member of the pneumococcal histidine-triad (Pht) protein family characterized by the presence of histidine-triads. PhtD is a 838 aa-molecule and carries 5 histidine triads (see MedImmune WO00/37105 SEQ ID NO: 4 for amino acid sequence and SEQ ID NO: 5 for DNA sequence). PhtD also contains a proline-rich region in the middle (amino acid position 348-380). PhtD has a 20 aa-N-terminal signal sequence. Preparation and purification of PhtD is described in WO2007/071710 (see Example 1b).

### Description of Transferred Material: SIV-p27 Lot PE04MY1901

### Buffer:

DPBS (NaCl 136.87 mM, KCl 2.68 mM, Na₂HPO₄ 8.03 mM, KH₂PO₄1.47 mM)

### Recombinant Protein:

SIV p27 from SIV mac 251 is described in WO2009/077436 (SEQ ID No. 19).

### Preparation:

E. coli expression, extraction in 50 mM TRIS-HCl pH 8.0, BLUE Trisacryl Plus, ammonium sulfate precipitation, DPBS recovery, DPBS dialysis, Acticlean Etox, concentration, Acticlean Etox, concentration.

### Protein Characteristics:

Molecular Weight 27477 Da
Molar Extinction coefficient: 38010±5%
   1A(280)=0.72 mg/ml
Isoelectric Point: 5.77
   Preparation of the Vaccine Composition with Adjuvant

1. AS01B

1.1 Preparation of the 2-Fold Concentrated AS01B

Phosphate Buffer Saline pH6.1 when diluted 10 times was added to water for injection to reach respectively 10 mM phosphate and 140 mM NaCl concentrations in the final formulation. Concentrated liposomes (made of DOPC, cholesterol and MPL) were added to QS21 and mixed 15 min at room temperature by magnetic stirring. The mixture made of liposomes and QS21 was added to the diluted buffer and mixed 30 min at room temperature by magnetic stirring. The pH was checked so as to be around 6.0. In the two fold concentrated adjuvant, the concentration of the QS21 was 200 μg/ml and the concentration of MPL was 200 μg/ml

1.2 Preparation of the Final Formulations

PhtD or dPly at 180 or 60 μg/ml in AS01B

The formulations were prepared extemporaneously according the following sequence: Water For Injection+Saline Buffer pH6.1 when 10 fold diluted+2-fold concentrated adjuvant, 5 min mixing on an orbital shaking table at room temperature,+antigen (quantities were added in order to reach final concentrations of 180 μg/ml or 60 μg/ml), 5 min mixing on an orbital shaking table at room temperature,

### AS01B Alone

The formulation was prepared extemporaneously according the following sequence: Water For Injection+Saline Buffer pH6.1 when 10 fold diluted+2-fold concentrated adjuvant, 2×5 min mixing on an orbital shaking table at room temperature.

2. AS15

2.1 Preparation of the 2-Fold Concentrated AS15

Phosphate Buffer Saline pH6.1 when diluted 10 times was added to water for injection to reach respectively 10 mM phosphate and NaCl 140 mM concentrations in the final formulation. Concentrated liposomes (made of DOPC, cholesterol and MPL) were added to QS21 and mixed 15 min at room temperature by magnetic stirring. The mixture made of liposomes and QS21 was added to the diluted buffer and mixed 30 min at room temperature by magnetic stirring. CpG was added in order to be at 1680 μg/ml in the concentrated adjuvant. The adjuvant was mixed 15 min at room temperature by magnetic stirring. The pH was checked so as to be around 6.0.

In the two fold concentrated adjuvant, the concentration of QS21 is 200 μg/ml of MPL was 200 μg/ml and of CpG was 1680 μg/ml.

2.2 Preparation of the Final Formulations

PhtD or dPly or p27gag at 1000 μg/ml in AS15

The formulations were prepared extemporaneously according the following sequence: Water For Injection+Saline Buffer pH6.1 when 10 fold diluted+2-fold concentrated adjuvant 5 min mixing on an orbital shaking table at room temperature,+antigen (quantities are added in order to reach a final concentration of 100 μg/ml), 5 min mixing on an orbital shaking table at room temperature.

3. AS03B

3.1 Preparation of the Final Formulation

PhtD or dPly at 1800 μg/ml or 600 μg/ml in AS03B

The formulations were prepared extemporaneously according the following sequence: Water For Injection+Saline Buffer pH6.8 when 10 fold diluted+SB62 oil in water emulsion (250 μl/ml final formulation), 5 min mixing on an orbital shaking table at room temperature,+antigen (quantities were added in order to reach final concentrations of 180 μg/ml or 60 μg/ml), 5 min mixing on an orbital shaking table at room temperature,

### AS03B Alone

The formulation was prepared extemporaneously according the following sequence: Water For Injection+Saline Buffer pH6.8 when 10 fold diluted+SB62 oil in water emulsion (250 μl/ml final formulation), 2×5 min mixing on an orbital shaking table at room temperature.

### T Cell Responses

Briefly, peripheral blood lymphocytes (PBLs) from 28 mice/group and 14 mice/group for positive controls were collected and pooled (4 or 2 pools of 7 mice/group). A red blood cells lysis was performed before plating the cells on round 96-well plates at 1 million cells per well. The cells were then re-stimulated in vitro with a pool of overlapping 15 mers peptides (at 1 μg/ml/peptide containing the two antibodies CD49d and CD28) for 2 hours. Cells remaining in the medium (no peptide stimulation) were used as negative controls for background responses. Two hours after the co-culture with the peptide pool, Brefeldin A was added to the wells (to inhibit cytokine excretion) and the cells were further incubated overnight at 37° C. with 5% CO₂. The cells were subsequently stained for the following markers: CD4, CD8, IL-2, IFN-γ, IL13 and IL17. Samples were analyzed by Flow cytometry.

### Intracellular Cytokine Staining

Following the antigen restimulation step, PBLs are incubated overnight at 37° C. in presence of Brefeldin (1 μg/ml) at 37° C. to inhibit cytokine secretion.

IFN-γ/IL17/IL3 or IL5/IL2/CD4/CD8 staining was performed as follows: cell suspensions were washed, resuspended in 50 μl of PBS 1% FCS containing 2% Fc blocking (anti-CD16/32) reagent (1/50).

After 10 min incubation at 4° C., 50 μl of a mixture of anti-CD4 pacific Blue (1/50) and anti-CD8 perCp-Cy5.5 (1/50) was added and incubated 30 min at 4° C. After a washing in PBS 1% FCS, cells were permeabilized by resuspending in 200 μl of Cytofix-Cytoperm (kit BD™) and incubated 20 min at 4° C. Cells were then washed with Perm Wash (kit BD™) and resuspended with 50 μl of a mix of anti-IFN-γ APC (1/50)+anti-IL-2-FITC (1/50)+anti-IL13 or IL5-PE (1/50)+anti-IL17-Alexa 700 (1/50) diluted in Perm wash. After an incubation of 1 h, cells were washed with BD™ stabilizing-fixative solution (BD Biosciences). Samples analysis were performed by FACS. Live cells were gated (FCS/SSC) and acquisition was performed on ≈10 000 CD8 cells. The percentage of IFN-γ+ or IL17+ or IL3 or IL5+ or IL2 were calculated on CD4 and CD8+ gated populations.

### Cell Mediated Immunity was Evaluated by Cytokine Flow Cytometry (CFC)

Peripheral blood antigen specific CD4 and CD8 T cells can be restimulated in vitro to produce IFNγ, IL2, IL13, IL17 if incubated with their corresponding antigen. Consequently, antigen specific CD4 and CD8 T cells can be enumerated by flow cytometry following conventional immunofluorescence labelling of cellular phenotype as well as intracellular cytokines production. In the present study, PhtD and dPly proteins as well as peptides derived from these specific streptococcus proteins were used as antigen to restimulate specific T cells. Results were expressed as a frequency of cytokines positive CD4 or CD8 T cell within CD4 or CD8 cell sub-population.

### Quantification of IgG:

Purified PhtD and Ply was coated respectively at 1 and 4 μg/ml in PBS on high-binding micotitre plates (NUNC Maxisorp) 2 hours at 37° C. The mouse anti-sera were diluted and then further twofold dilutions were made in microplates and incubated at RT for 30 min with agitation. After washing, the bound antibodies were detected using Jackson ImmunoLaboratories Inc. peroxidase-conjugated affinipure Goat Anti-Mouse IgG (H+L) (ref:115-035-003) diluted 1/2500 in PBS-Tween 0.05%. These detection antibodies were incubated for 30 min at room temperature with agitation. After washing, the color was developed using 4 mg OPD+5 μl H₂O₂ per 10 ml PH4.5 0.1M citrate buffer for 15 minutes in the dark at room temperature. The reaction was stopped with 50 μl 1N HCl, and the optical density (OD) was read at 490-620 nm. The level of anti-PhtD and anti-dPly IgG present in the serum samples is determined by comparison to the curve of the reference and was expressed in μg/ml.

### Summary of Results and Conclusions

Antigen-specific T cell responses induced by dPly/PhtD in AS01B or AS03B were evaluated in blood post-III in C57BL6 mice. A high antigen-specific T cell response was induced with dPly/PhtD in AS01B whereas a low or no response was observed with AS03B. AS01B induces mainly IFN-γ secreting CD4+ T cells (Th1). AS01B induces mainly Th17 specific to dPly 7 days after the third immunization whereas barely detectable Th17 response can be induced with AS03B. AS15/sivP27 or dPly/AS15 were used as positive controls for Th17 induction.

The antibody IgG responses induced by AS01B for the two proteins were also higher than with AS03B.

### Example 2

### Evaluation of the Adjuvants in the Lethal Challenge Model (MF1 with 4CDC Strain)

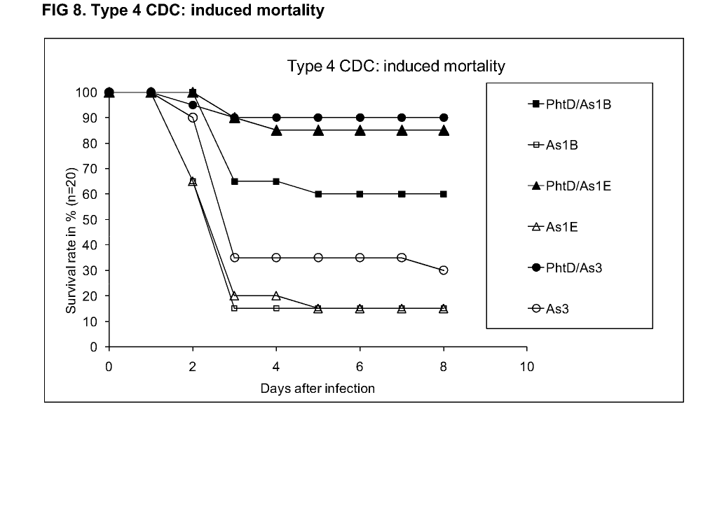

Different adjuvants were evaluated in the lethal challenge model. OF1 female mice (4 week old) were immunized intramuscularly (IM) on days 0 and 14 with 2 doses of 3 μg/50 μl PhtD antigen formulated with different adjuvant system (AS01B, AS01E and AS03). Control mice were vaccinated with adjuvant system alone. Mice were subsequently challenged intranasally with 5×106 CFU of S. pneumoniae type 4CDC. Mortality was recorded for 8 days. The results are shown in FIG. 8.

The protection against the strain 4CDC was almost complete (around 90%) with AS01E, and AS03 combined with PhtD. A significant difference (between PhtD/AS (vaccinated mice) and the AS alone (negative control)) was observed for all adjuvants. Nevertheless, the best difference between vaccinated mice and the corresponding negative control was observed for AS01E.

### Evaluation of the Adjuvant in the Lung Colonisation Model

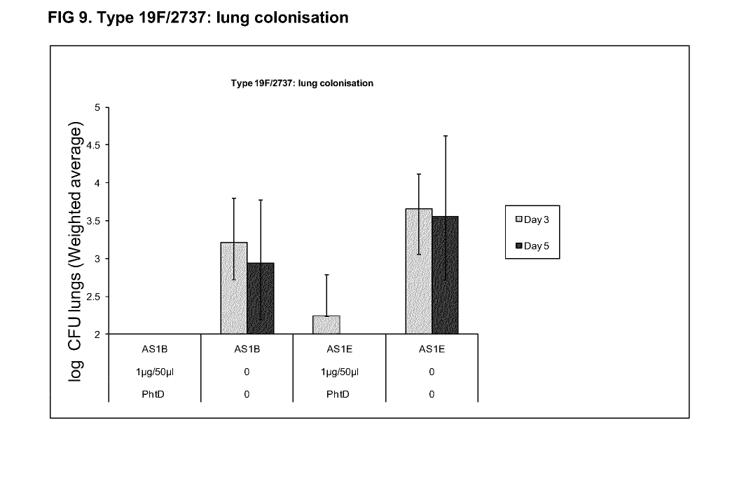

Two adjuvants were evaluated in the lung colonisation model. CBAJ female mice were immunized intramuscularly (IM) on days 0, 14 and 28 with PhtD formulated with different adjuvant system (AS01B, AS01E). Control mice were vaccinated with adjuvant system alone. Mice were subsequently challenged intranasally with 2×107 CFU of S. pneumoniae type 19F/2737. Bacterial load was measured by colony counting in lungs collected 3 and 5 days post-challenge. The results are shown in FIG. 9.

A significant protection was induced in this model after immunization with PhtD either adjuvanted with AS01B or AS01E compared to the negative control groups that only received the corresponding adjuvant alone.

1. An immunogenic composition comprising at least one unconjugated Streptococcus pneumoniae protein selected from: pneumolysin and member(s) of the Polyhistidine Triad family; and an adjuvant comprising QS21, monophosphoryl lipid A (MPL), phospholipid and sterol, presented in the form of a liposome.

2. An immunogenic composition as defined in claim 1 wherein the ratio of Streptococcus pneumoniae protein:monophosphoryl lipid A (MPL) is 0.05:1 to 3:1 (w/w).

3. An immunogenic composition as defined in claims 1-2 wherein the ratio of Streptococcus pneumoniae protein:QS21 is 0.05:1 to 3:1 (w/w).

4. An immunogenic composition as defined in claims 1-3 comprising 5 to 60, 45 to 55, 5 to 20, or 20 to 30 μg (e.g. 20, 25, 30, 35, 40, 45 or 50 μg) monophosphoryl lipid A (MPL).

5. An immunogenic composition as defined in claims 1-4 comprising 5 to 60, 45 to 55, 5 to 20, or 20 to 30 μg (e.g. 20, 25, 30, 35, 40, 45 or 50 μg) QS21.

6. An immunogenic composition as defined in claims 1-5 comprising 0.1 to 10 mg, 0.2 to 7, 0.3 to 5, 0.4 to 2, or 0.5 to 1 mg (e.g. 0.4 to 0.6, 0.9 to 1.1, 0.5 or 1 mg) phospholipid.

7. An immunogenic composition as defined in claims 1-6 comprising 0.025 to 2.5, 0.05 to 1.5, 0.075 to 0.75, 0.1 to 0.3, or 0.125 to 0.25 mg (e.g. 0.2 to 0.3, 0.1 to 0.15, 0.25 or 0.125 mg) sterol.

8. An immunogenic composition as defined in claims 1-7 wherein the monophosphoryl lipid A (MPL) is 3-O-Deacylated monophosphoryl lipid A (3D-MPL).

9. An immunogenic composition as defined in claim 8 wherein the amount of 3D-MPL is 50 μg, per human dose.

10. An immunogenic composition as defined in claims 1-9 wherein the amount of QS21 is 50 μg, per human dose.

11. An immunogenic composition as defined in claims 1-10 wherein phospholipid is dioleoylphosphatidylcholine (DOPC).

12. An immunogenic composition as defined in claim 11 wherein the amount of DOPC is 1000 μg, per human dose.

13. An immunogenic composition as defined in claims 1-12 wherein sterol is cholesterol.

14. An immunogenic composition as defined in claim 13 wherein the amount of cholesterol is 250 μg, per human dose.

15. An immunogenic composition as defined in claims 1-14 capable of invoking a cytolytic T cell response in a mammal.

16. An immunogenic composition as defined in claims 1-15 capable of stimulating interferon γ production.

17. An immunogenic composition as defined in claims 1-16 capable of stimulating IL-17 production.

18. An immunogenic composition as defined in claims 1-17 wherein the pneumolysin is detoxified pneumolysin (dPly).

19. An immunogenic composition as defined in claim 18 wherein the pneumolysin has been chemically detoxified.

20. An immunogenic composition as defined in claim 18 or 19 wherein the pneumolysin has been genetically detoxified.

21. The immunogenic composition as defined in claims 1-20 comprising 3 to 90, 3 to 20, to 40 or 40 to 70 μg (e.g. 10, 30 or 60 μg) unconjugated pneumococcal pneumolysin, per human dose.

22. The immunogenic composition as defined in claims 1-21 wherein the member of the Polyhistidine Triad family is PhtD.

23. The immunogenic composition as defined in claim 22 wherein the PhtD comprises an amino acid sequence at least 90% identical to the sequence at amino acids 21-838 of Sequence ID No. 4 of WO00/37105.

24. The immunogenic composition as defined in claim 22 wherein the PhtD has an amino acid sequence at least 90% identical to the sequence at amino acids 21-838 of Sequence ID No. 4 of WO00/37105.

25. The immunogenic composition as defined in claim 22 wherein the PhtD has an amino acid sequence comprising amino acids 21 to 838 of Sequence ID NO: 4 of WO00/37105.

26. The immunogenic composition as defined in claim 22 wherein PhtD has an amino acid sequence comprising at least 10 contiguous amino acids from Sequence ID No. 4 of WO00/37105.

27. The immunogenic composition as defined in claims 1-26 comprising 3 to 90, 3 to 20, to 40 or 40 to 70 μg (e.g. 10, 30 or 60 μg) unconjugated PhtD, per human dose.

28. The immunogenic composition as defined in claims 1-27 comprising unconjugated pneumolysin and unconjugated pneumococcal PhtD.

29. An immunogenic composition as defined in claims 1-28 comprising one or more further antigens.

30. An immunogenic composition as defined in claims 1-28 comprising one or more S. pneumoniae capsular saccharides.

31. An immunogenic composition as defined in any of the preceding claims wherein the dose volume is between 0.4 and 1.5 ml

32. An immunogenic composition as defined in claim 31 wherein said dose volume is 0.5 ml.

33. A vaccine comprising the immunogenic composition as defined in claims 1-32.

34. A method of making a vaccine as claimed in claim 33 comprising the steps of mixing the unconjugated Streptococcus pneumoniae protein with the adjuvant composition.

35. A method of eliciting an immune response by immunising a mammal with the immunogenic composition of claims 1-32.

36. A method of treating or preventing a disease caused by Streptococcus pneumoniae infection comprising administering to a patient suffering from or susceptible to Streptococcus pneumoniae infection an immunogenic composition as defined in any one of claims 1-32.

37. A method of treating or preventing AE COPD comprising administering to a patient suffering from or susceptible to AE COPD an immunogenic composition as defined in any one of claims 1-32.

38. A method of treating or preventing a disease caused by Streptococcus pneumoniae infection comprising intramuscularly administering to a subject in need thereof comprising administering to said subject an immunogenic composition as defined in any one of claims 1-32.

39. A method of treating or preventing a disease caused by Streptococcus pneumoniae infection comprising intramuscularly administering to a human in need thereof comprising administering to said human an immunogenic composition as defined in any one of claims 1-32.

40. An immunogenic composition as defined in any one of claims 1-32 for use in treating or preventing a disease caused by Streptococcus pneumoniae infection.

41. An immunogenic composition as defined in any one of claims 1-32 for use in treating or preventing AE COPD.

42. Use of an immunogenic composition as defined in any of claims 1-32 in the manufacture of a medicament for use in treating or preventing a disease caused by Streptococcus pneumoniae infection.

43. Use of an immunogenic composition as defined in any of claims 1-32 in the manufacture of an intramuscular vaccine for use in treating or preventing a disease caused by Streptococcus pneumoniae infection.

44. Use of an immunogenic composition as defined in any of claims 1-32 in the manufacture of a medicament for use in treating or preventing AE COPD.
